(19) United States
(12) Patent Application Publication
(10) Pub. No.: US 2019/0224092 A1
(43) Pub. Date: Jul. 25, 2019

(54) ALCOHOL-FREE ORAL CARE COMPOSITIONS AND METHODS

(71) Applicant: Avaria Health and Beauty Corp., Cambridge (CA)

(72) Inventors: Keith Burk, Cambridge (CA); Philip Hudson, Cambridge (CA); Fan Wang, Cambridge (CA)

(73) Assignee: Avaria Health and Beauty Corp., Cambridge (CA)

(21) Appl. No.: 16/258,386

(22) Filed: Jan. 25, 2019

(30) Foreign Application Priority Data
Jan. 25, 2018 (CA) 2992871

(51) Int. Cl. A61K 8/34; A61K 8/92; A61K 8/73; A61K 8/49; A61Q 11/00

(52) U.S. Cl. CPC A61K 8/345; A61K 8/922; A61K 8/731; A61K 2800/5922; A61Q 11/00; A61K 2800/48; A61K 2800/49; A61K 8/4926

(57) ABSTRACT

In one embodiment, a water-based, alcohol-free, oral care composition comprises antimicrobial agents, bacteriostatic agents, solubilizing agents, humectants and flavouring agents. The pleasant sensory properties are derived from its active ingredients and flavouring agents from natural sources. This oral rinse composition can be made by different methods. This oral rinse product has high antibacterial efficacy and positive organoleptic properties that encourage more frequent use to reduce bad breath and promote oral rehydration.

### TECHNICAL FIELD

The present disclosure relates to oral care compositions, and in particular to alcohol-free oral care compositions comprising antimicrobial and bacteriostatic agents, and which are pleasant tasting.

### BACKGROUND

Oral hygiene and health are important for well-being. Poor oral hygiene can lead to long term adverse effects such as halitosis, plaque formation and tooth and gum decay. Using an antibacterial oral rinse product can help improve oral health. Presently, many mass market oral rinse products are based on ethyl alcohol and phenolic compounds which can create a sensation of burning and discomfort after holding for approximately 15 to 20 seconds in the oral cavity, causing expectoration long before the effective bacterial kill time. This burning can also discourage regular use. Additionally, many oral rinse products contain anti-bacterial agents which typically have an undesirable flavour, therefore requiring artificial flavouring agents and solubilizing agents to mask their bitter flavour. Typical solubilizing agents include silicone and surfactants, many of which also exhibit an undesirable flavour. Suitable flavouring agents can come from natural sources and/or artificial sources.

### SUMMARY OF PARTICULAR EMBODIMENTS

It will be appreciated by those skilled in the art that other variations of the embodiments described below may also be practiced without departing from the scope of the invention. Further note, these embodiments, and other embodiments of the present invention will become more fully apparent from a review of the description and claims which follow.

In one embodiment, the present disclosure relates to a water-based, alcohol-free, oral care composition comprising antimicrobial agents, bacteriostatic agents, solubilizing agents, humectants and flavouring agents. This oral care composition has high antibacterial efficacy and positive organoleptic properties that encourage more frequent use to reduce bad breath and promote oral rehydration.

In another embodiment, the present disclosure relates to an alcohol-free, water-based oral rinse solution comprising multiple active ingredients. In another particular embodiment, the composition has pleasant palatability which allows users to hold same in their oral cavity for a sufficient bacterial-killing time, thereby encouraging long-term, repetitive usage.

In one embodiment, the composition eliminates typical adverse user experiences of an oral rinse product which can prevent appropriate and continued use (these include unpleasant flavouring, burning sensation, and post-application dryness).

In yet another embodiment, the composition includes a primary antibacterial agent and multiple bacteriostatic agents which also function as humectants and flavouring agents. In one particular embodiment, the composition is further flavoured with multiple flavouring agents from natural sources dispersed with solubilizing agents.

In one embodiment, the composition contains thickeners which contribute to its film forming property in the oral cavity to reduce dryness and enhance antimicrobial efficacy. In one embodiment, the composition of the present invention demonstrates superior antibacterial performance. The composition of the present invention is non-irritating, and can also be used by aging and/or vulnerable populations who have higher tendency to develop dry mouth (xerostomia) and other conditions that are related to oral bacteria, to reduce their risk of progressive deterioration of oral health.

### DETAIL DESCRIPTION OF EXAMPLE EMBODIMENTS

In this respect, before explaining at least one embodiment of the invention in detail, it is to be understood that the invention is not limited in its application to the details of construction and the arrangements of the components outlined in the following description or illustrated in the drawings. The invention is capable of other embodiments and of being practiced and carried out in various ways. Also, it is to be understood that the phraseology and terminology employed herein are for the purpose of description and should not be regarded as limiting. In particular, all terms used herein are used in accordance with their ordinary meanings unless the context or definition clearly indicates otherwise. Also, unless indicated otherwise except within the claims the use of “or” includes “and” and vice-versa. Non-limiting terms are not to be construed as limiting unless expressly stated or the context clearly indicates otherwise (for example, “including”, “having”, “characterized by” and “comprising” typically indicate “including without limitation”). Singular forms included in the claims such as “a”, “an” and “the” include the plural reference unless expressly stated or the context clearly indicates otherwise. Further, the stated features and/or configurations or embodiments thereof the suggested intent may be applied as seen fit to certain operating conditions or environments by one experienced in the field of art.

The present disclosure provides oral care compositions, methods of manufacture for the composition, and method of administration or application to, or use with, a human or other animal subject. The oral care compositions of the present disclosure are intended to be retained as a suitable amount in the oral cavity for a time sufficient to reduce bacteria count. Materials to be used in the compositions of the present disclosure must be food, pharmaceutically and/or cosmetically acceptable.

In one embodiment, the oral care compositions of the present disclosure combine a water-soluble antibacterial agent, xylitol, glycerin, thickeners, solubilizing agents, and flavouring agents. Water-soluble anti-bacterial agents that helps kill oral bacteria represent the preferable anti-bacterial agent in this composition. Oral care actives, such as xylitol and glycerin are bacteriostatic so that they prolong the bacterial reduction effect after application. Flavouring agents mask the bitter taste of the antibacterial agents thereby improving the sensory properties of the composition. These ingredients are dispersed in water with glycerin as the carrier. In one embodiment, after application, the oral rinse leaves a film on the surface of teeth and the oral cavity, which provides prolonged anti-bacterial effect and enhances hydration inside the oral cavity. The embodiments described herein do not require the use of ethanol and other C1-C6 monohydric alcohols as ingredients, which in turn reduces the sensation of irritation while holding the current oral rinse in the mouth, thereby promoting sufficient holding time to reduce oral bacterial count.

The term “oral rinse” in the present invention refers to oral compositions that are substantially liquid in character, including but not limited to a mouthwash, spray, or rinse.

Xylitol is a non-cariogenic sugar alcohol, and it has been used as a sweetener, a humectant, and an anti-bacterial agent. Xylitol is bacteriostatic (i.e. bacteria cannot reproduce) and has specific effects on oral microflora related to dental caries. Xylitol may cause a disturbance in the metabolism of fermentable carbohydrates by S. mutans and thereby decreases plaque formation and reduces plaque adhesion to the pellicle. Xylitol functions as a humectant at this usage level. Xylitol also contributes to the sweetness and viscosity of the oral rinse product. In one embodiment, xylitol may be present in the composition as an anti-caries agent at 5% to 40% by weight in the present invention.

In one embodiment, the composition of the present disclosure contains one or more antibacterial agents, preferably water-soluble agents. Some suitable examples include, but are not limited to, zinc ions, copper (II) compounds, phthalic acid and salts, hexetidine, octenidine, sanguinarine, benzalkonium chloride, domiphen bromide, alkylpyridinium chlorides, iodine, sulfonamides, bisbiguanides, plant-based compounds such as magnolia extract, grapeseed extract, menthol, geraniol, citral, eucalyptol, antibiotics such as amoxicillin, amoxicillin with clavulanic acid, tetracycline, doxycycline, minocycline, metronidazole, neomycin, kanamycin and clindamycin and the like. Antimicrobial agents may be present in the composition at 0.1% to 2% by weight.

Cetylpyridinium chloride (CPC) is a water-soluble antimicrobial agent. CPC is germicidal against a broad range of bacteria and has been used in other oral care products. CPC has a bitter taste which can potentially contribute to undesirable sensory properties. CPC may be present in the composition at 0.01% to 0.2% by weight.

Glycerin functions as a solvent, a bacterial static agent and a humectant in the current invention. Glycerin also contributes to the sweetness and viscosity of the oral rinse product. Glycerin may be present in the composition at 5% to 40% by weight.

Orally-acceptable thickeners include, without limitation, carbomers, carrageenans, cellulosic polymers such as hydroxyethylcellulose, carboxymethylcellulose, hydroxypropyl methylcellulose (HPMC) and salts, agar, natural gums such as alginates, guar gum, karaya gum, xanthan, gum Arabic and tragacanth, colloidal magnesium aluminum silicate, colloidal silica and the like. One or more thickening agents are optionally present in a total amount of 0.01% to about 5% by weight.

A thickener that does not have an electrostatic charge and does not have adverse interactions with other ingredients is preferred because it can provide the product with a desirable appearance and flow property. A thixotropic rheological behaviour is also beneficial because it could provide a desirable flow property when swishing the product inside the oral cavity. The viscosity of the oral rinse may be between 30 and 1000 centipoise (cP) at room temperature.

The sweetness from xylitol and glycerin can help overcome the unpleasant flavour and/or taste of the other antimicrobial agents.

The composition of the present disclosure can be flavoured with one or more hydrophobic and hydrophilic flavouring agents. The flavouring agents can be natural or synthetic, but preferably emanate from natural sources. The flavouring agents can be, but are not limited to, mint (e.g. peppermint essential oil, spearmint essential oil), citrus (e.g. lemon, lime, orange, tangerine and grapefruit), honey, ginger, green tea, phenol, eucalyptol, and thymol. The combination of the flavouring agents may enhance the desirable taste of the composition.

The cumulative concentration of the flavouring agents may be at 0.01% to 4% by weight.

In one embodiment, the composition uses water as its carrier and most of the flavouring agents are hydrophobic, therefore, solubilizing agents are required to disperse the flavouring agents. Solubilizing agents that can be employed should not interact with and/or bind to other ingredients used. Solubilizing agents should also not create an excessive amount of foam when swishing in the oral cavity and should not have a soapy taste. Solubilizing agents that maintain a clear appearance in the current invention are preferred. Solubilizing agents can be, but are not limited to, polysorbates, (block) copolymers, (poly)acrylic acids, fatty acid esters, polar lipids, polyglycerol fatty esters, polyethylene glycols (PEGs) and betaines. The accumulative concentration of the solubilizing agents may be present at 0.01% to 5% by weight.

In one embodiment of the present invention, xylitol and glycerin are the two active ingredients that contribute to the sweetness of the composition. Additional sweeteners may be added to the composition for the purpose of further enhancing the flavour and/or taste of the composition.

Optional ingredients, such as chelating agents, colouring agents, analgesic, anesthetic and anti-inflammatory agents can also be included in the composition.

The employment of thickening agents enhances the film-forming properties of the composition, to extend contact time of active ingredients with the oral mucosa. Xylitol, glycerin, and thickening agents increase the viscosity of a solution, thereby contributing to the mouth-coating and hydration effect and reducing the dryness of the oral cavity.

The antimicrobial properties of the compositions of the present disclosure have been shown to be significantly superior to a leading national brand oral care product containing ethanol and phenolic compounds. The compositions of the present disclosure demonstrate superiority in antibacterial performance when compared with an alcohol-based leading national brand oral rinse product.

Oral cavity swab samples have been collected to test the antimicrobial efficacy of the compositions described herein. Swab samples were taken from oral cavities treated with an alcohol-based leading national brand, the current alcohol-free oral rinse or sterile saline control for 30 seconds. These swabs were then plated and incubated. The total plate count (TPC) after treatment using the leading national brand was 2.11×10⁴ colony forming units/ml (cfu/ml). The TPC after treatment with our formulation was 1.01×10² cfu/ml. The oral cavity treated with the current invention has a 200-fold lower total bacterial count than that treated with a national leading brand oral care product.

In a method of inhibiting oral microorganisms, a user contacts his or her tissue and/or teeth in the oral cavity with the oral care composition described herein for a sufficient time to reduce the number of microorganisms in the oral cavity.

Methods are described herein for reducing bad breath of a user, comprising administering a safe and effective amount, 10-15 mL, of a mouth rinse composition (as described herein) to the oral cavity of the subject. The subject keeps the oral rinse composition in contact with the surface of the oral cavity while swishing for 30 seconds. The oral rinse composition can also be applied to the oral cavity as a leave-on spray. The oral rinse prepared in accordance with the present invention is intended to be used regularly in the oral cavity, at least one time daily and may be used repeatedly as required. The present invention may also be used for the improvement of dry mouth (xerostomia) and overall systemic health characterized by a reduction in risk of infections.

The oral rinse composition can be manufactured based on the following methods.

The oral rinse compositions of the present invention may be prepared by suitably mixing the ingredients. Thickeners are first dispersed through a powder chute into 0% to 100% of water heated to above 65° C., then another 0% to 100% of water is added cold, recirculating through an in-line powder disperser and allowing to hydrate. Water-soluble anti-bacterial agents are then added. Once the thickening agents are fully hydrated, xylitol may be added via the chute and then glycerin. Once all the xylitol crystals are dissolved and the solution is clear, disperse the solubilizing agent and flavouring agents and recirculate while shear mixing. Examples of antibacterial agents include phenolic compounds such as β-naphthol, thymol, chlorothymol, amyl-, hexyl-, heptyl- and octylphenol, hexylresorcinol, hexachlorophene, and phenol; quaternary ammonium compounds such as quaternary morpholinium alkyl sulfates, cetylpyridinium chloride, alkyldimethyl benzylammonium chloride, and alkyltrimethyl ammonium halides; and miscellaneous antibacterial compounds such as benzoic acid, formaldehyde, potassium chlorate, tyrothricin, gramicidin, iodine, sodium perborate, and urea peroxide, however, many of these compounds have a disagreeable taste, or are only antibacterially effective at levels considered unsafe for human use.

The oral rinse compositions described herein may be prepared by suitably mixing the ingredients in one phase through a sequence. Thickeners are firstly dispersed into 0 to 100% of water heated to above 65° C. in the main vessel, then another 0 to 100% of water is added cold. Water-soluble anti-bacterial agent is then added. Recirculate the solution through an in-line homogenizer to allow hydration. Add xylitol and glycerin into the vessel and continue recirculation. Disperse the solubilizing agent and flavouring agents in the batch and continue to shear mix until homogeneous.

The oral rinse compositions described herein may be prepared by suitably mixing the ingredients in two different phases through a sequence. Thickeners are firstly dispersed into 0 to 100% of water heated to above 65° C. in the first vessel, then another 0 to 100% of water is added cold, allowing hydration or full dispersion of thickeners. Water-soluble anti-bacterial agent is then added into the vessel holding dispersed thickeners. This vessel is the thickener phase. In a separate vessel, disperse the solubilizing agent and flavouring agents into 0-100% of cold or hot water, and recirculate the solution through an in-line homogenizer until homogeneous. This vessel is called the flavour phase. Combine the contents of the thickener phase and the flavour phase. Add xylitol and glycerin into the vessel, and continue recirculation through the in-line homogenizer until all xylitol is dissolved and the solution is homogeneous.

The oral rinse compositions described herein may be prepared by suitably mixing the ingredients in three phases through a sequence. Thickeners are firstly dispersed into 0 to 100% of water heated to above 65° C. in the main vessel, then another 0 to 100% of water is added cold, allowing hydration or full dispersion of thickeners. Water-soluble anti-bacterial agent is then added into the vessel holding dispersed thickeners. This vessel is the thickener phase. In a separate vessel, disperse the solubilizing agent and flavouring agents into 0 to 100% of cold or hot water, and recirculate the solution through an in-line homogenizer until homogeneous. This vessel is called the flavour phase. In a third vessel, disperse xylitol and glycerin into 0 to 100% of cold or hot water, mixing until all the xylitol is dissolved. This is called the xylitol phase. Combine the contents in the thickener phase, the flavour phase, and the xylitol phase, and continue recirculation through the in-line homogenizer until homogeneous.

The oral rinse compositions described herein may be prepared by suitably mixing the ingredients through a sequence that involves a secondary loop of the product. Thickeners are firstly dispersed into 0 to 100% of water heated to above 65° C. in the primary vessel, then another 0 to 100% of water is added cold, allowing hydration of thickeners. Water-soluble anti-bacterial agent, xylitol, and glycerin are added into the vessel at the same time. Once the thickening agent is fully hydrated, 5 to 20% of the content is transferred into a separate vessel. The solubilizing agent and flavouring agents are added into the separate vessel for homogenization, using a jet mixer, a shear mixer or an in-line homogenizer. Once homogenized, the contents of this vessel can be transferred back to the primary vessel and mix homogeneously. The secondary loop allows efficient mixing of the flavouring agents and reduces viscosity loss introduced by shear through homogenization.

### EXAMPLES

The invention is illustrated in the following examples.

### Example 1: Formulation of the Oral Rinse Product

Water 75%

Xylitol 12%

Glycerin 12%

Polysorbate 20 0.75%

Peppermint oil 0.1%

Hydroxyethylcellulose 0.1%

Cetylpyridinium chloride 0.05%

### Example 2: Method of Making the Ingredients Presented in Example 1 using a Powder Disperser

1. Disperse hydroxyethylcellulose through a powder chute into 10% of total weight of water heated to above 65° C., then another 65% of total weight of water cold;

2. Recirculating the suspension through an in-line powder disperser and allowed to hydrate;

3. Add cetylpyridinium chloride into the vessel;

4. Once the hydroxyethylcellulose is fully hydrated, add xylitol and glycerin via the chute of the powder disperser;

5. Once all the xylitol crystals are dissolved and the solution is clear, disperse Polysorbate 20 and peppermint oil while shear mixing until the solution is homogeneous.

### Example 3: Method of Making the Ingredients Presented in Example 1 using an In-Line Homogenizer Through One-Phase Mixing

1. Disperse hydroxyethylcellulose into 10% of total weight of water heated to above 65° C., then the another 65% of total weight of water cold;

2. Recirculating the suspension through an in-line homogenizer and allow to hydrate;

3. Disperse cetylpyridinium chloride into the vessel;

4. Add xylitol and glycerin into the vessel and continue recirculation;

5. Once all the xylitol crystals are dissolved and the solution is clear, disperse Polysorbate 20 and peppermint oil while keep recirculation through the in-line shear mixer until the solution is clear.

### Example 4: Method of making the ingredients presented in Example 1 through a secondary loop using a jet mixer.

1. Disperse hydroxyethylcellulose through a powder chute into 10% of total weight of water heated to above 65° C., then another 65% of total weight of water cold;

2. Disperse cetylpyridinium chloride, xylitol and glycerin into the vessel;

3. Mix the suspension with a jet mixer to allow hydroxyethylcellose to hydrate and to allow xylitol to dissolve;

4. Transfer 25% of the content into a second vessel;

5. Disperse Polysorbate 20 and peppermint oil into the second vessel;

6. Homogenize the second vessel using a jet mixer;

7. Once a homogenous emulsion has formed, transfer content in the second vessel back to the primary vessel;

8. Mix the primary vessel with a jet mixer until homogenous.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, functions, operations, or steps, any of these embodiments may include any modification, combination or permutation of any of the components, elements, functions, operations, or steps described or illustrated anywhere herein that a person having ordinary skill in the art would comprehend. All such modifications, combinations and permutations are believed to be within the sphere and scope of the invention as defined by the claims appended hereto.

What is claimed is:

1. An oral care composition, comprising xylitol, glycerin, thickeners and water, wherein the xylitol and glycerin are synergistically effective to inhibit bacteria while presenting positive organoleptic properties.

2. The oral care composition according to claim 1, containing about 5% to 40% by weight of xylitol.

3. The oral care composition according to claim 1, containing about 5% to 40% by weight of glycerin.

4. The oral care composition according to claim 1, containing about 0.01% to about 5% by weight of thickeners.

5. The oral care composition according to claim 1, further containing about 0.01% to 0.2% by weight of an anti-bacterial agent.

6. The oral care composition according to claim 1, wherein the thickener is selected from the group consisting of carbomers, carrageenans, cellulosic polymers such as hydroxyethylcellulose, carboxymethylcellulose, hydroxypropyl methylcellulose (HPMC) and salts, natural gums such as alginates, karaya, xanthan, gum Arabic, acacia, guar gum, agar, and tragacanth, colloidal magnesium aluminum silicate, and colloidal silica, and mixtures thereof.

7. The oral care composition according to claim 1, further containing about 0.01% to 4% by weight of flavouring agents.

8. The oral care composition according to claim 7, wherein the flavoring agent is selected from the group consisting of hydrophobic and hydrophobic compounds, and mixtures thereof.

9. The oral care composition according to claim 1, further containing about 0.01% to 5% by weight of solubilizing agents.

10. The oral care composition according to claim 1, wherein the solubilizing agent is selected from the group consisting of polysorbates, (block)copolymers, (poly)acrylic acids, fatty acid esters, polar lipids, polyglycerol fatty esters, polyethylene glycols (PEGs) and betaines, and mixtures thereof.

11. An oral care composition consisting essentially of by weight of the total weight of the composition: about 5% to 40% by weight of xylitol, about 5% to 40% by weight of glycerin, about 0.01% to 4% by weight of flavouring agents, about 0.01% to 5% by weight of solubilizing agents, and water q.s. to 100%.

12. An oral care composition for inhibiting bacteria and presenting pleasing organoleptic properties, comprising an effective amount of xylitol, an effective amount of glycerin, and a suitable carrier.
